(19) United States
(12) Patent Application Publication
(10) Pub. No.: US 2026/0162774 A1
(43) Pub. Date: Jun. 11, 2026

(54) METHOD FOR PREDICTING PRODUCTION STABILITY OF CLONAL CELL LINES

(71) Applicant: GLAXOSMITHKLINE INTELLECTUAL PROPERTY DEVELOPMENT LIMITED, Stevenage (GB)

(72) Inventors: Antonio BENEDETTI, Stevenage (GB); Pierantonio FACCO, Padova (IT); Ruth Christine ROWLAND-JONES, Stevenage (GB)

(73) Assignee: GLAXOSMITHKLINE INTELLECTUAL PROPERTY DEVELOPMENT LIMITED, Stevenage (GB)

(21) Appl. No.: 19/126,682

(22) PCT Filed: Nov. 14, 2023
(86) PCT No.: PCT/EP2023/081700

§ 371 Date: May 2, 2025

Related U.S. Application Data
(60) Provisional application No. 63/425,886, filed on Nov. 16, 2022.

(51) Int. Cl. G16B 40/30; C07K 16/00; G16B 40/20

(52) U.S. Cl. CPC G16B 40/30; C07K 16/00; G16B 40/20

(57) ABSTRACT

The invention relates to a method for selecting a clonal cell line for use in production of a therapeutic protein, the method comprising the steps of measuring, for a plurality of clonal cell lines, a product concentration of each clonal cell line; determining, based on the product concentration, product concentration profile data of each clonal cell line, inputting the product concentration profile data into a learning model comprising a modelling framework, wherein the modelling framework comprises multivariate latent variable modelling, multiway analysis structure, and evolving models structure; generating, using the learning model, an output indicating a production stability of each clonal cell line; and selecting, based on the output, a clonal cell line for use in production of a therapeutic protein product. The invention also relates to a system for determining clonal cell line production stability or selecting a clonal cell line.

### FIELD OF THE INVENTION

The invention relates to methods of developing cell lines for biopharmaceutical protein production, particularly methods and systems for determining or predicting production stability of a clonal cell line, and methods and systems for selecting a clonal cell line.

### BACKGROUND TO THE INVENTION

Fully human antibodies can be obtained using a variety of methods, for example using yeast-based libraries or transgenic animals (e.g. mice) that are capable of producing repertoires of human antibodies. Yeast presenting human antibodies on their surface that bind to an antigen of interest can be selected using FACS (Fluorescence-Activated Cell Sorting) based methods or by capture on beads using labelled antigens. Transgenic animals that have been modified to express human immunoglobulin genes can be immunised with an antigen of interest and antigen-specific human antibodies isolated using B-cell sorting techniques. Human antibodies produced using these techniques can then be characterised for desired properties such as affinity, developability and selectivity.

Mammalian cell lines are used as cell factories to create therapeutic protein-producing clonal cell lines. A nucleic acid sequence encoding the protein of interest is cloned into an expression vector and subsequently transfected into the host cell line. Transfected pools are bulked, single cell sorted, and outgrowth of these single cell sorted, clonal cell lines are then assessed for their production of the protein of interest. Clonal cell lines are ranked based on their product concentration and undergo a series of triage events until around 50 clonal cell lines are selected to enter a production stability assessment.

Examples of such mammalian cell lines include murine myeloma cells (NSO), baby hamster kidney cells (BHK), human embryonic kidney cells (HEK293) and Chinese hamster ovary cells (CHO), with over 80% of currently approved recombinant proteins being produced in CHO cells (Butler & Spearman, 2014; Walsh, 2018). Reasons for the widespread use of CHO cells include their relative ease in receiving and expressing exogenous DNA, rapid growth rates, the reliability of their protein folding machinery, their adaptability to serum-free suspension culture, and their ability to produce proteins with human-like post-translational modifications.

However, cell lines such as CHO cells are known to have a high level of chromosomal and genomic heterogeneity (Wurm & Wurm, 2017). As a result of this genetic plasticity, cell lines may demonstrate production instability whereby a decrease in recombinant protein quantity and quality is observed during long periods of culture (Dahodwala & Lee, 2019).

Consequently, biopharmaceutical manufacturers must demonstrate to regulators that cells used for therapeutic product production maintain stable protein quality over time. Cell line production stability trials are typically designed with 3 or 4 evaluation points spanning 60 to 80 cell line generations. Cell lines tested in these stability trials are typically defined as stable when they display a decrease of less than 30% in recombinant protein concentration (Dahodwala & Lee, 2019). Under this criterion, it has been estimated that up to 63% of all CHO cell lines evaluated in production stability trials are classified as unstable (Dahodwala & Lee, 2019).

Without maintenance of product concentration throughout the manufacturing period, process yield can have a significant impact on timelines as manufacturing schedules are typically booked up to at least a year in advance. As such, unexpectedly low product yields can lead to repeat manufacture runs having an enormous impact on scheduling and a knock-on effect on product distribution. These stability trials thus represent an important yet time-consuming and resource-intensive endeavour for manufacturers.

Modelling techniques have typically been applied to industrial process monitoring in the context of the field of soft sensors (Ramaker et al, 2005; Camacho et al, 2008; Gunther et al, 2008). Soft sensors may incorporate online measurements of process variables such as temperature, pH and dissolved oxygen (DO) into models to predict an output variable. For example, Gunther et al used online process variable measurements to predict product titre. However, satisfactory prediction of the important quality of cell line production stability has not been achieved in the field of soft sensors. As such, cell line production stability trials remain a time-consuming and costly undertaking.

Accordingly, there is a current need for methods which reduce the amount of time and resources expended during cell line production stability trials.

### SUMMARY OF THE INVENTION

According to a first aspect, there is provided a method for selecting a clonal cell line for use in production of a therapeutic protein, comprising: measuring, for a plurality of clonal cell lines, a product concentration of each clonal cell line; determining, based on the product concentration, product concentration profile data of each clonal cell line; inputting the product concentration profile data into a learning model comprising a modelling framework; wherein the modelling framework comprises multivariate latent variable modelling, multiway analysis structure, and evolving models structure; generating, using the learning model, an output indicating a production stability of each clonal cell line; and selecting, based on the output, a clonal cell line for use in production of a therapeutic protein product.

According to a second aspect, there is provided a method for producing a therapeutic protein, comprising: measuring, for a plurality of clonal cell lines, a product concentration of each clonal cell line; determining, based on the product concentration, product concentration profile data of each clonal cell line; inputting the product concentration profile data into a learning model comprising a modelling framework; wherein the modelling framework comprises multivariate latent variable modelling, multiway analysis structure, and evolving models structure; generating, using the learning model, an output indicating a production stability of each clonal cell line; selecting, based on the output, a clonal cell line for use in production of a therapeutic protein product.

According to a further aspect, there is provided a system for determining clonal cell line production stability or selecting a clonal cell line, the system comprising (a) an input for receiving clonal cell line product concentration profile data; (b) a learning model to determine clonal cell line production stability or select a clonal cell line, the learning model comprising a modelling framework comprising multivariate latent variable modelling, multiway analysis structure, and evolving models structure; (c) one or more processors for processing clonal cell line product concentration profile data with the learning model; and (d) an output to provide an indication of clonal cell line production stability based on the processing of clonal cell line product concentration profile data by the learning model.

According to a further aspect, there is provided a computer program comprising instructions which, when the program is executed by a computer or data processor(s), cause the computer to perform operations according to the method described herein.

According to a further aspect, there is provided a computer-readable medium comprising instructions which, when executed by a computer or data processor(s), cause the computer to perform operations according to the method described herein.

According to a further aspect, there is provided a computer-readable data carrier having stored thereon the computer program described herein.

The methods and systems of the disclosure are advantageous in that they facilitate early robust prediction of clonal cell line production stability. In particular, the incorporation of clonal cell line product concentration profile measurements into the modelling framework described herein provides a technical advantage in that, in comparison to other methods, clonal cell line production stability can be predicted with greater accuracy and efficiency to assist selection of clonal cell lines during the biopharmaceutical development process. By applying this methodology at an early stage of the cell line development (CLD) period, it is possible to triage clonal cell lines predicted to be productionally unstable earlier, thereby increasing CLD capacity and reducing chemistry, manufacturing and controls (CMC) timelines.

The details of one or more embodiments of the invention are set forth in the accompanying description below. Other features, objects, and advantages of the invention will be apparent from the description and from the claims.

### DESCRIPTION OF DRAWINGS/FIGURES

FIG. 1 shows an exemplary flowchart for selecting a clonal cell line for use in production of a therapeutic protein.

FIG. 2 shows a schematic of the batch-wise unfolding to deal with multi-way arrays in the multivariate classification of clonal cell line production stability.

FIG. 3 shows a schematic of the evolving multiway classification procedure.

FIG. 4 shows an exemplary process for production stability classification of clonal cell lines using a trained modelling framework incorporating labelled calibration datasets.

FIG. 5 shows 3D score plots for the unsupervised evolving multi-way principal component analysis (EMPCA) models of Set A where the squares are the stable clonal cell lines and the circles are the unstable clonal cell lines. The principal component space represents the dimensionality reduction resulted from the multivariate analysis of the titre features, but with no formal class supervision (unsupervised).

FIG. 6 shows 3D score plots for the unsupervised evolving multi-way principal component analysis (EMPCA) models of Set B where the squares are the stable clonal cell lines and the circles are the unstable clonal cell lines. The principal component space represents the dimensionality reduction resulted from the multivariate analysis of the titre features, but with no formal class supervision (unsupervised).

FIG. 7 shows the Variables Important for Prediction (VIPs) of the PLS-DA model generated after production run #3 as discussed in Example 4. Variables with a high VIP score may be important for the prediction of clonal cell line production stability.

### DETAILED DESCRIPTION OF THE INVENTION

Before discussing particular embodiments with reference to the accompanying figures, the following description of embodiments is provided.

In a first aspect there is provided a method for selecting a clonal cell line for use in production of a therapeutic protein, comprising: measuring, for a plurality of clonal cell lines, a product concentration of each clonal cell line; determining, based on the product concentration, product concentration profile data of each clonal cell line; inputting the product concentration profile data into a learning model comprising a modelling framework; wherein the modelling framework comprises multivariate latent variable modelling, multiway analysis structure, and evolving models structure; generating, using the learning model, an output indicating a production stability of each clonal cell line; and selecting, based on the output, a clonal cell line for use in generation of a therapeutic protein product.

The multivariate latent variable modelling may comprise at least one of Projection to Latent Structures (PLS) and Principal Component Analysis (PCA). The PLS may comprise at least one of PLS-Discriminant Analysis (PLS-DA), PLS-Support-Vector Machines (PLS-SVM), PLS-Neural Networks (PLS-NN), PLS-Logistic Regression (PLS-LR), PLS-k-Nearest Neighbours (PLS-KNN), PLS-Decision Tree (PLS-DT), PLS-Naïve Bayes (PLS-NB), PLS-Random Forest (PLS-RF) and PLS-Gradient Boost (PLS-GB).

The product concentration profile data may comprise one or more of mean product concentration, standard deviation, skewness, kurtosis, differential, maximum product concentration and maximum-minimum gradient. The product concentration may comprise the concentration of a monoclonal antibody.

Measuring, for a plurality of clonal cell lines, a product concentration of each clonal cell line may comprise measuring a product concentration of each clonal cell line across a plurality of production runs. The clonal cell line product concentration may be measured for 2, 3, or 4 production runs. The clonal cell line product concentration may be measured for 2 production runs. The clonal cell line product concentration may be measured for 3 production runs. The clonal cell line product concentration may be measured for 4 production runs. The clonal cell line product concentration may be measured for up to 150 generations. The method may further comprise obtaining a generation number distribution for each production run.

The evolving models structure may comprise analysing the clonal cell line product concentration profile of sequential production runs.

The clonal cell line may be a mammalian cell line. The clonal cell line may be a CHO cell line.

The clonal cell line product concentration may be measured at multiple bioreactor scales. The clonal cell line product concentration may be measured at a bioreactor scale of 15 mL.

Unless defined otherwise, all technical and scientific terms used herein have the same meaning as is commonly understood by one of skill in the art to which this invention belongs. All patents and publications referred to herein are incorporated by reference in their entirety.

The term “comprising” encompasses “including” or “consisting” e.g. a composition “comprising” X may consist exclusively of X or may include something additional e.g. X+Y.

The term “consisting essentially of” limits the scope of the feature to the specified materials or steps and those that do not materially affect the basic characteristic(s) of the claimed feature.

The term “consisting of” excludes the presence of any additional component(s).

The term “about” in relation to a numerical value x means, for example, x±10%, 5%, 2% or 1%.

The term “clonal cell line” or “clone” as used herein refers to an isolated host cell comprising a gene of interest. Here isolation of the host cell means separation from other host cells, using techniques known in the art, such as FACS (fluorescence-activated cell sorting) or dilution cloning. A clonal cell line may undergo a therapeutic protein production stability analysis as described herein, during which the isolated clonal cell line will be grown in a cell culture. Cells grown in said cell culture will share a common ancestry to the respective clonal cell line.

The terms “clonal cell line production stability” or “production stability” as used herein refers to the stability of production of therapeutic protein by a clonal cell line, that is to say production of a consistent product concentration or titre of therapeutic protein over 50 to 150 generations. Consistent product concentration or titre may be defined as <30% drop in therapeutic protein product concentration or titre.

The terms “clonal cell line product concentration”, “product concentration”, “titre” or “productivity titre” as used herein refers to the amount of therapeutic protein produced by a clonal cell line, that is to say the concentration of therapeutic protein produced by a clonal cell line. The concentration of therapeutic proteins produced by a cell line may be measured using techniques such as ELISA, HPLC, Western blot, immunoassay, detection of protein biological activity, FACS analysis, fluorescence microscopy, direct detection of a fluorescent protein by FACS analysis, spectrophotometry or other techniques known in the art. The terms “clonal cell line product concentration profile”, “clonal cell line product concentration profile data”, “titre profile”, “product concentration profile” or “product concentration profile data” as used herein refer to the set of variables (features) calculated from the measured product concentration data for each clonal cell line. For example, the product concentration profile of a clonal cell line may comprise one or more of mean product concentration, standard deviation, skewness, kurtosis, differential, maximum product concentration and the maximum-minimum gradient.

The term “production run” as used herein refers to the process by which the protein encoded by the gene of interest produced by the clonal cell line is expressed, whereby one or more clonal cell lines in individual vessels undergo feeding, growth and therapeutic protein production phases. A production stability trial comprises multiple production runs.

A “batch” may be defined as a single vessel for a clonal cell line going through the full feeding, growth and therapeutic production phases of a production run. As such, a “production run” comprises multiple batches of separate clonal cell lines going through the full feeding, growth and therapeutic protein production phases for production of a protein product. During a production stability trial, multiple clonal cell lines are assessed for production stability, and each clonal cell line is present in a separate batch of each production run.

The term “generation number” or “generation” as used herein refers to the number of times a clonal cell line has doubled. For example, a generation number of 80 generations means that a clonal cell line has doubled 80 times.

The term “product” as used herein refer to a protein produced by a clonal cell line. Accordingly, the terms “protein product”, “cell line product” and “therapeutic protein product” are used interchangeably with the term “product” for the purposes of the described invention. “Therapeutic protein production” refers to the process of production of a therapeutic protein product by a clonal cell line.

The term “antibody” as used herein refers to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing.

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H₂L₂ structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or λ light chain. The majority of antibodies in the serum belong to the IgG class, there are four isotypes of human IgG (IgG1, IgG2, IgG3 and IgG4), the sequences of which differ mainly in their hinge region.

This application relates to methods and systems for assessing the production stability of clonal cell lines, in order to select a clonal cell line for use in production of a therapeutic protein product. Production stability assessment of a clonal cell line is essential. In order for a clonal cell line to progress to the manufacturing stage, it must produce a consistent amount of therapeutic protein across the manufacturing window (typically 3 to 6 months). A standard production stability assessment involves scaling up the clonal cell lines and inoculating production vessels across a 3 to 6 month period to reflect the length of time of the manufacturing window. To calculate production stability, product concentration measurements of each production run are taken and percent product concentration change across the time series is calculated. Generally, clonal cell lines which are able to maintain their protein expression to within 30% of their original peak product concentration during the stability assessment are considered stable.

In an industry setting, for each therapeutic protein, around 50 clonal cell lines are typically progressed to production stability assessment, from which a single clonal cell line, that is deemed manufacturable, will be selected. As such, cell line development (CLD) processes for the manufacturing of a biopharmaceutical product require significant investment of time and resources. Data analysis may involve considerable time expenditure and inconsistency in approach which can lead to selection of unstable clones and/or rejection of the best performing clones.

High-throughput automated platforms of Advanced Microscale Bioreactors with 10-15 mL working volume (AMBR 15) represent an opportunity to standardize the experimental workflow whilst collecting and systematically storing a huge amount of data. Data availability and automation pave the way for the development of an appropriate data modelling framework to exploit the full capabilities of the acquired measurements with the final purpose of improving stability trial design, reducing the preventable experimental effort and enhancing the stability characterization of future clonal cell lines.

Variables such as temperature, pH, dissolved oxygen (DO) and air flow may be monitored online during industrial processes. Soft sensors may utilise these monitored variables as input data in a model that proceeds to predict an output target measurement. Such soft sensors have been developed when online analysers are not available or economically feasible for process variables of interest. For example, models using online monitoring of variables including temperature, pH and DO have been used to predict endpoint offline measurements of product titre (Gunther et al, 2008).

In the current disclosure, the inventors have surprisingly found that product titre measurements may be incorporated as input data into a modelling framework described herein to accurately predict production stability of a clonal cell line. In contrast, incorporation of other variables measured during online monitoring of industrial processes into the modelling framework was not predictive of later clonal cell line production stability. Using the method described herein, the timelines for selecting productionally stable clonal cell lines during cell line development may be shortened by accurately predicting clonal cell line production stability at an earlier stage.

Production stability trial data may be harnessed to develop models which facilitate early robust prediction of clonal cell line production stability. By analysing the product concentration profiles of consecutive production runs during stability trials, an early ‘fingerprint’ may be identified in clonal cell lines, which is predictive of later production stability. The developed methodology utilizes an advanced data-driven modelling approach that combines multivariate analysis diagnostic capabilities with machine learning classification techniques to assess the production stability. By applying this methodology at an early stage of the 3 to 6 month CLD period, it may be possible to triage clonal cell lines predicted to be productionally unstable earlier, thereby increasing CLD capacity and reducing chemistry, manufacturing and controls (CMC) timelines. When clonal cell lines are predicted or determined to be stable or unstable by the method described herein, the stable clonal cell lines may be selected for implementation in subsequent recombinant therapeutic protein production processes.

The sensitivity of the method for predicting or determining production stability is related to the accuracy of stable clonal cell line predictions. The specificity of the method for predicting or determining production stability is related to the accuracy of unstable clonal cell line predictions. Because the aim of the method is to predict, determine or select stable clonal cell lines during CLD for therapeutic protein production, high sensitivity is preferred over specificity.

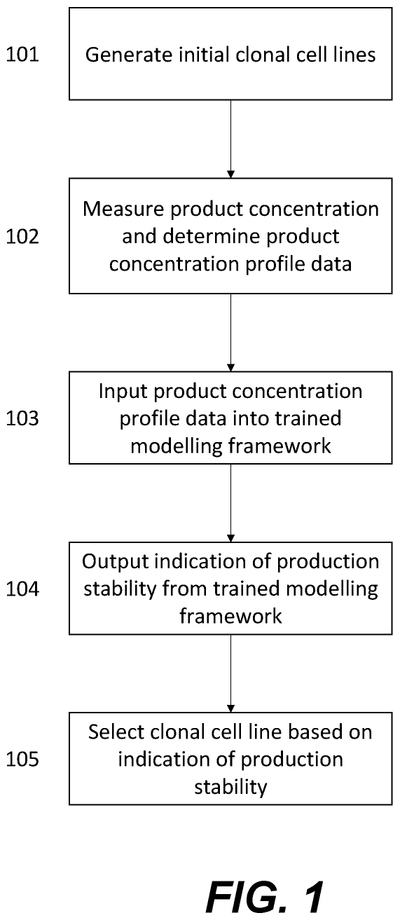

FIG. 1 illustrates an exemplary method for selecting a clonal cell line for use in production of a therapeutic protein. In step 101, a plurality of clonal cell lines are generated. This step 101 may comprise cloning a nucleic acid sequence encoding the protein of interest into an expression vector and transfecting this sequence into a host cell line. A host cell expressing the gene of interest may then be isolated through separation from other host cells, using techniques known in the art, such as FACS (fluorescence-activated cell sorting) or dilution cloning. The person skilled in the art will appreciate that the process by which a set of clonal cell lines may be generated may be accomplished through any technique known in the art and used for this purpose. The method for selecting a clonal cell line may comprise the step of generating a plurality of clonal cell lines. The initial set of clonal cell lines may then undergo a production run, wherein each set of clonal cell lines may be fed and grown such that they express therapeutic protein encoded by the gene of interest.

In step 102, the concentration of therapeutic proteins expressed by each clonal cell line of the set of clonal cell lines—also referred to as the productivity titre of each clonal cell line—may be measured. This product concentration may be measured using techniques such as ELISA, HPLC, Western blot, immunoassay, detection of protein biological activity, FACS analysis, fluorescence microscopy, direct detection of a fluorescent protein by FACS analysis, spectrophotometry or other techniques known in the art. Multiple product concentration measurements may be taken within each production run of a stability trial. The measurement of a product concentration of each clonal cell line may be automated. Further data may be calculated based on the productivity titre, including (but not limited to) the mean product concentration, standard deviation (standard deviation of the product concentration profile), skewness (degree of asymmetry), kurtosis (sharpness), differential (difference between two consecutive measurements for all of the product concentration profile datapoints), maximum product concentration (maximum of the product concentration profile) and maximum-minimum gradient (gradient of the line between the maximum product concentration and the minimum of the product concentration profile). Any of these variables, alone or in combination, may be referred to as clonal cell line product concentration profile”, “clonal cell line product concentration profile data”, “titre profile”, “product concentration profile” or “product concentration profile data.” The differential variable values are calculated from an array of n−1 cells where n is the number of the datapoints for a product concentration profile of a production run. As such, multiple differentials can be calculated from one product concentration profile. For example, differential6 is defined as the differential between the sixth and seventh product concentration datapoint measured in a product concentration profile. Stability trials for assessment of clonal cell line production stability may occur at multiple bioreactor scales. For example, product concentration profile data may be generated from production runs at a scale or vessel size of 384-well plate, 96-well plate, 48-well plate, 24-well plate, 12-well plate, 6-well plate, T25, T75, T150, AMBR 15 or AMBR 250. The person skilled in the art will further appreciate that steps 101 and 102 may be repeated multiple times in each iteration of the process shown in FIG. 1, and that each iteration may comprise multiple production runs of an initial set of clonal cell lines. Production stability trials for clonal cell lines typically comprise at least 3 consecutive production runs, often 4 or more consecutive production runs over a 4 to 6-month period. The method may comprise determining product concentration profile data of 2, 3, 4, 5, 6, 7, 8, 9 or 10 production runs. That is to say, product concentration profile data may be determined after each production run of 2, 3, 4, 5, 6, 7, 8, 9 or 10 production runs. The method may comprise determining product concentration profile data of 2 or more, 3 or more or 4 or more production runs. The method may comprise determining product concentration profile data of 2 production runs. The method may comprise determining product concentration profile data of 3 production runs. The method may comprise determining product concentration profile data of 4 production runs. Stability trials may consider a window of 0 to 150 clonal cell line generations, whereby each consecutive production run is performed at an increasing generation number for each clonal cell line. The method may comprise determining product concentration profile data of clonal cell lines having a generation number of at least 15, at least 20, at least 25, at least 30, at least 35, at least 40, at least 45 or at least 50. The method may comprise determining product concentration profile data of clonal cell lines having a generation number of up to 50, up to 60, up to 70, up to 80, up to 90, up to 100, up to 110, up to 120, up to 130, up to 140 or up to 150.

In step 103, the product concentration profile data gathered and/or calculated in step 102 may be provided to a trained modelling framework. The modelling framework utilised may comprise multivariate latent variable modelling, multiway analysis structure, and evolving models structure. Collectively, this modelling framework may be defined as an Evolving Multiway Projection to Latent Structures (EMPLS) or as an Evolving Multiway Principal Component Analysis (EMPCA). The multivariate latent variable modelling may be used to develop the classification model which enables the modelling framework to discriminate stable from unstable clonal cell lines and to give a probabilistic attribution of the stability class. Examples of multivariate latent variable modelling include Projection to Latent Structures (PLS) (Brereton & Lloyd, 2014), also known as Partial Least Squares, and Principal Component Analysis (PCA). The multivariate latent variable modelling may comprise PLS, PCA, EMPLS, EMPCA, Evolving Multiway Projection to Latent Structures Discriminant Analysis (EMPLS-DA), or any combination thereof.

In methods utilising EMPLS-DA, the methodology may comprise:
a multivariate latent variable classification method, i.e. Projection to Latent Structures-Discriminant Analysis (Brereton & Lloyd, 2014), to discriminate if a clonal cell line is either stable or unstable, also considering measurement correlation (cross-correlation, autocorrelation and correlation in time) and giving a probabilistic attribution of the stability class;
a multi-way methodology (Nomikos & MacGregor, 1994) to account for measurement differences across batches within a single experimental production run; and
an evolving modelling structure (Ramaker et al., 2005) to consider the progression in sequential stability trial production runs conducted at increasing generation number of the clones.

Projection to Latent structures (PLS) (Geladi & Kowalski, 1986; Wold et al., 1983) is a multivariate regression technique. PLS deals with a huge amount of correlated input data stored in a matrix of regressors X [N×M] of N experiments made on different clonal cell lines where M variables are measured and relates them to the C corresponding response variables collected in a matrix Y [N×C]. Data are typically “auto-scaled”, i.e. mean centered and scaled to unit variance to avoid the effect of different measurement units. PLS reduces the dimension of the original X space by finding A orthogonal (i.e., independent) latent variables (LVs) which explain the inputs X variance that is most predictive for the response Y. Accordingly, PLS decomposes X and Y as:

$$X = {TP}^{T} + E = {\sum }_{a = 1}^{A} {t}_{a} {p}_{a}^{T} + E \qquad ( 1 )$$

$$Y = {UQ}^{T} + F = {\sum }_{a = 1}^{A} {u}_{a} {q}_{a}^{T} + F \qquad ( 2 )$$

$${u}_{a} = {b}_{a} {t}_{a} \qquad ( 3 )$$

where T [N×A] and U [N×A] are the scores matrices of X and Y, respectively; P [M×A] and Q [C×A] are the loadings matrices of X and Y, respectively, whose columns are pₐ and qₐ; superscript T indicates the transpose; E [N×M] and F [N×P] are residuals of X and Y, respectively, minimized in a least-squares sense to properly fit the calibration data; tₐ and uₐ are columns of T and U, respectively, and are linearly related through the regression coefficients bₐ. The scores are the projection of the original data into the reduced space of the LVs and identify the relation among the N experiments on different clones; the loadings are the director cosines of the LVs with respect to the original variables and identify the correlation among variables; the residual matrices include the non-systematic part of the datasets (when the number of LVs A is properly selected). Usually, a small number of LVs A<<min(N, M, P) is sufficient to retain the important information in both X and Y variability, no matter how large the dimension of these matrices is. Cross-validation (Wold, 1978) is usually used to determine the optimal number of LVs.

When measurements of the M regressors are available for a new observation xᵢ, the respective responses can be estimated by projecting xᵢ onto the model space according to:

$$\hat{y} = {x}_{i} {WQ}^{T} \qquad ( 4 )$$

where ŷ is the vector of the estimated responses and W [M×A] is the weight matrix for the projection of xᵢ onto the latent space.

PLS can be effectively utilized for multivariate classification as the so-called Projection to Latent Structures-Discriminant Analysis (PLS-DA) (Brereton & Lloyd, 2014). In such cases the matrix Y is composed of categorical variables (and not continuous variables). In particular, in binary classification problems the Y variable is expressed as a “dummy” variable in which 1 indicates that a sample belongs to that class and 0 that a sample does not belong to that class.

In this case, two classes are present: the stable clonal cell line (yᵢ=1) and the unstable clonal cell lines (yᵢ=0). The class attribution for a new sample xᵢ can be obtained from Equation 4. However, an estimation error eᵢ:

$${e}_{i} = {y}_{i} - {\hat{y}}_{i} \qquad ( 5 )$$

is always present for the class attribution. For this reason, a distribution of the estimated values ŷᵢ around the real value yᵢ (that we recall is 1 if i belongs to the stable class, 0 otherwise) is calculated from the all calibration data (whose class is known a priori). The probability density function (PDF) is calculated assuming that the distribution of all the ŷᵢ∀i=1 to N of the calibration dataset around the real value yᵢ is Gaussian. Furthermore, the intersection among the PDFs of the two classes, stable and unstable, provides the threshold th value which discriminates if the clonal cell line i whose estimation is ŷᵢ is attributed to the stable class or not. Furthermore, cumulative density functions (CDFs) are calculated from the abovementioned PDFs for all the classes in such a way as that the inversion of the CDFs provides the probability P of being associated to each class. In summary, not only through Equation 4 it is possible to attribute every new clonal cell line i to a class (i.e., stable or unstable) based on the fact that ŷᵢ>th or ŷᵢ<th, but it is also possible to understand the degree of confidence that is associated to this decision, namely the probability P that clonal cell line i actually belongs to either of the two classes (P_{i, stable/i, unstable}). To simplify the concept of the two class probabilities into a single estimated value, an arbitrary definition of “confidence” in prediction is defined as:

$${C}_{i} = \vert {P}_{i , stable} - {P}_{i , unstable} \vert \qquad ( 6 )$$

This definition of “confidence” in prediction ensures that the following scenarios are appropriately described:
if P_{i, stable}>>P_{i, unstable}, or vice versa, the difference within the two values is high, and therefore the class with the higher probability is also predicted with high confidence;
if P_{i, stable}>P_{i, unstable}, or vice versa, the difference within the two values is moderate, and therefore the class with the higher probability is also predicted with moderate confidence;
if P_{i, stable}≈P_{i, unstable}, the difference is low, and therefore the confidence is low with little importance on either a stable or unstable prediction.

The multivariate latent variable modelling of the method may be supervised or unsupervised. Supervised modelling is performed using labelled datasets, i.e. there is prior knowledge of the data output which trains algorithms to classify data and predict outcomes. Unsupervised modelling is performed without labelled output variables such that the algorithm is able to identify hidden patterns in datasets. The use of unsupervised modelling may be used to determine whether differences in the CLD platforms and processes affect the predictive ability of a modelling framework trained on a calibration dataset from another platform or process. Unsupervised modelling may also be used to determine whether the modelling framework discriminates between stable and unstable clonal cell lines, that is to say that the unsupervised modelling framework determines whether the product concentration profile data from early production runs display a natural fingerprint of the final production stability of clonal cell lines. Unsupervised modelling analysis of product concentration profile data of different designs, processes or platforms may therefore allow understanding of which production run, in the series of production runs within a production stability trial, the production stability discrimination is expressed as a fingerprint of the product concentration profile. Different processes and platforms which may be explored by unsupervised multivariate latent variable modelling include, but are not limited to, alternative cell line transfection systems, alterations in media composition, seeding conditions, and/or process parameters (e.g. pH or temperature ranges).

At step 104, the modelling framework may output an indication of the production stability of each clonal cell line for which an input has been provided. This indication may be a classification of each clonal cell line for which an input has been provided as productionally stable, or unstable. This classification may be reached using one or more classification methods as part of the multivariate latent variable modelling of the methods described herein. Examples of classification methods include, but are not limited to, Discriminant Analysis (DA), Support-Vector Machines (SVM), Neural Networks (NN), Logistic Regression (LR), k-nearest Neighbours (KNN), Decision Tree (DT), Naïve Bayes (NB), Random Forest (RF) or Gradient Boosting (GB). Therefore, in one example, the multivariate latent variable modelling of the methods described herein may comprise PLS-Discriminant Analysis (PLS-DA). According to other examples, the PLS may comprise PLS-DA, PLS-SVM, PLS-NN, PLS-LR, PLS-KNN, PLS-DT, PLS-NB, PLS-RF, PLS-GB or any combination thereof.

This step may be performed through calculation of the Probability Density Functions (PDFs) for input product concentration profile data. The intersection between the Probability Density Functions (PDFs) of the stable and unstable classes provides the threshold value which discriminates whether a clonal cell line is attributed to the stable class or not. If the probability value for a particular clonal cell line is greater than the threshold value, the clonal cell line will be attributed to the stable class. The attribution of clonal cell lines to the stable or unstable classes facilitates the prediction, determination or selection of clonal cell lines which are productionally stable. The multivariate latent variable modelling may attribute one or more clonal cell lines to the stable class. The multivariate latent variable modelling may attribute one or more clonal cell lines to the unstable class.

Optionally, the probability of a clonal cell line belonging to the stable or unstable class may be assigned confidence values. If the difference between the probability values for a clonal cell line being stable and unstable is high, moderate, or low, the class with the higher probability is predicted with high, moderate or low confidence, respectively. The clonal cell line production stability may be assigned confidence values. The confidence value may be high, and therefore the class with the higher probability may be predicted with high confidence. A high confidence value may be defined as ≥0.9, ≥0.8, ≥0.7 or ≥0.6. A moderate confidence value may be defined as ≥0.5, ≥0.4 or ≥0.3. A low confidence value may be defined as ≤0.3, ≤0.2 or ≤0.1. The determined production stability of one or more clonal cell lines may be assigned high confidence values, moderate confidence values or low confidence values.

At step 105, a clonal cell line that has been indicated as productionally stable may be selected. The skilled person will appreciate that this step may comprise deselecting clonal cell lines that have been marked as unstable. The resulting clonal cell line may be selected for use in subsequent recombinant therapeutic protein production processes, and may be used to generate or produce therapeutic protein product.

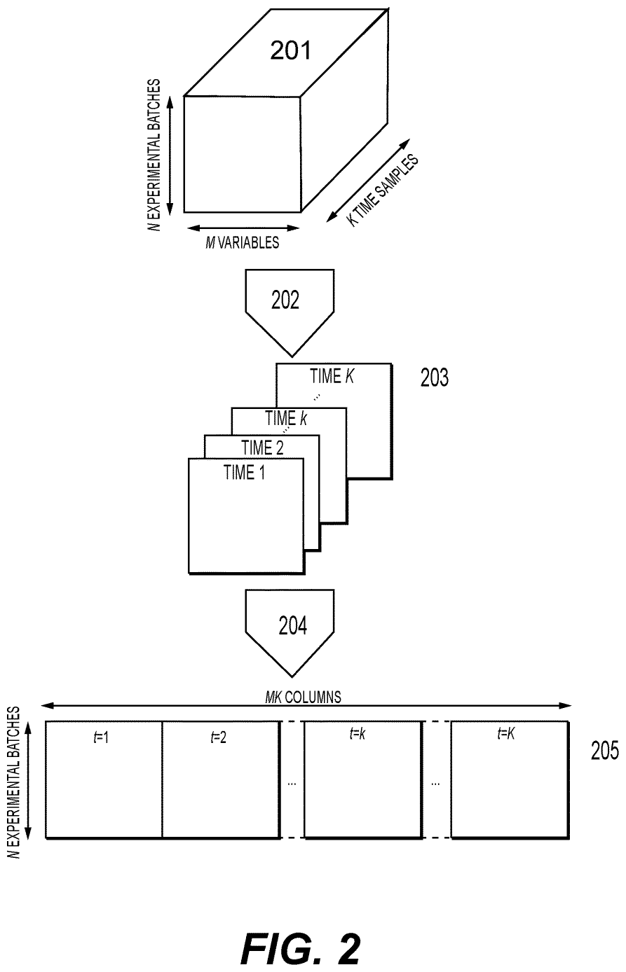

FIG. 2 provides an illustrative example of a multiway methodology of the kind that may be used in the invention, and particularly as described in connection with step 103 of FIG. 1. This methodology is particularly important when batch processes are considered, as the classification problem must consider a third dimension of the data, namely the time. To handle such cases, a modelling framework comprising multiway analysis structure may structure input data into a multidimensional array 201. The multidimensional arrays are batch data matrices of the product concentration profile variables, where the multidimensional array 201 is a three-way array with dimensions of the experimental batches (N), the product concentration profile variables (M) and the total number of time samples collected during an entire experimental production run (K). To process a three-dimensional array through PLS, array 201 is unfolded through process 202, to form a bi-dimensional one. This may be performed by cutting array 201 into vertical slices 203, which are then placed side by side in the so-called batch-wise unfolding fashion 204 (Nomikos & MacGregor, 1994), resulting in bi-dimensional matrix 205. PLS-DA can be applied to the resulting data matrix 205, which may be defined by dimensions [N×MK]. The batch-wise unfolding 204 requires that all batches have the same length K. Feature variables from the variable profiles, e.g. titre features from titre profiles, can be purposely defined instead of using the raw data (Meneghetti et al, 2016). The arrangement of the features will still follow the batch-wise fashion structure. PLS-DA applied to unfolded data matrix 205 is referred to as Multi-way PLS-DA (MPLS-DA).

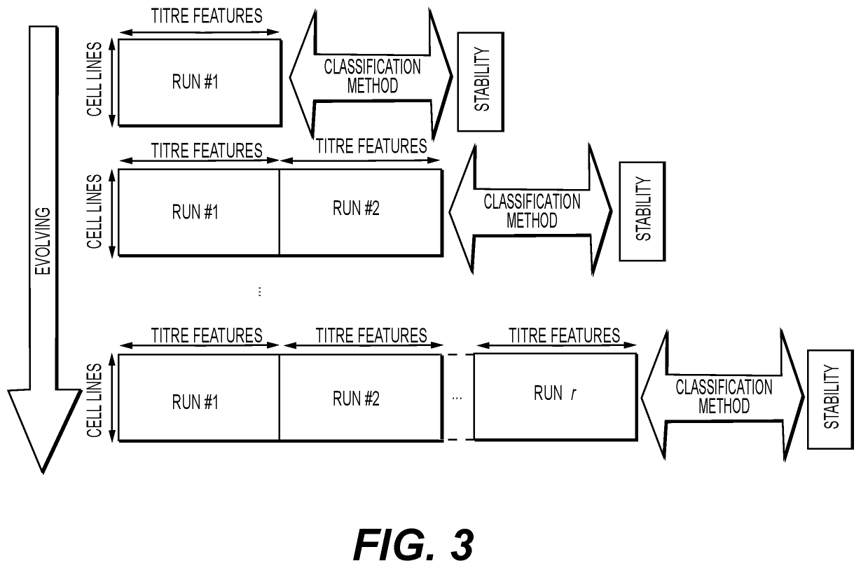

FIG. 3 provides an illustrative example of an evolving model structure such as might be used in the process of FIG. 1. Evolving models structure (Ramaker et al., 2005), also known as evolving modelling or evolving methodology, is a method which allows inclusion of data from the most recent production runs of a production stability trial in the classification model. When a production run is completed, the data collected therefrom may be included into the classification model concatenating horizontally the run's data (Ramaker et al., 2005). In this way, the incremental information coming from the most recent data may be exploited by the model in order to capture cross-correlation across production runs.

The “generation number” of a cell indicates the number of times the cell has doubled. The models are also built assuming that clonal cell lines associated to a certain production run should have a similar generation number across batches and project molecules. This allows flexibility in handling different number of production runs (e.g. independent models for projects where production runs <4 for a whole stability study). An assessment of the generation number distribution for each production run may allow alignment of production runs from different projects. The method described herein may further comprise assessing or obtaining a generation number distribution for each production run.

The evolving models structure, therefore, exploits the incremental information arising from sequential production runs and allows the capture of cross-correlation across production runs. When a production run is completed, the values of the product concentration profile variables within the production run are unfolded by multi-way analysis then included into the classification model generated by the multivariate latent variable modelling of the modelling framework. In this way, the classification models generated following each production run of a stability trial facilitate the classification of clonal cell lines as stable or unstable.

The use of evolving models structure may comprise analysing the clonal cell line product concentration profile of sequential production runs over increasing clonal cell line generations. The evolving models structure may comprise analysing the clonal cell line product concentration profile of 2 to 4 sequential production runs. The evolving models structure may comprise analysing the clonal cell line product concentration profile of 2 or more sequential production runs. The evolving models structure may comprise analysing the clonal cell line product concentration profile of 3 or more sequential production runs. PLS and PLS-DA applied to the multiway analysis structure and evolving models structure is referred to as EMPLS and EMPLS-DA, respectively. PCA applied to the multiway analysis structure and evolving models structure is referred to as a EMPCA.

### Variables Important for Prediction

Analysis of the product concentration variables in the methods described above may optionally allow determination of the Variables Important for Prediction (VIPs) in the prediction model. The VIP score represents the relative contribution that a given variable has to the classification of a clonal cell line as stable or unstable. Variables with high VIP scores may contribute relatively more to the prediction of production stability than variables with low VIP scores. As the assessment of VIPs is influenced by the calibration datasets used, the VIPs identified may differ between individual production runs or stability trials. By analysing multiple production runs of production stability trial datasets, it may optionally be possible to determine a subset of variables which have high VIP scores across production runs. Therefore, in some examples, the VIPs of the product concentration profile may comprise any of maximum, maximum-minimum gradient, standard deviation, differential or any combination thereof.

### Calibration Datasets

The modelling framework of the invention may use calibration datasets to train the algorithm to predict a stability class of one or more clonal cell lines. As such, previously acquired datasets are repurposed as calibration datasets to develop a model to predict production stability of clonal cell lines. The previously acquired calibration datasets are used as input datasets for the modelling framework to produce an output model which can classify clonal cell lines as stable or unstable. Analysing the clonal cell line product concentration profile may comprise use of a calibration dataset. Calibration datasets also allow assessment of the performance of the modelling framework, by comparing the predicted to the observed results. The calibration datasets used may comprise product concentration profile data from therapeutic protein-producing clonal cell lines. The calibration datasets may comprise product concentration profile data comprising one or more of mean product concentration, standard deviation, skewness, kurtosis, differential, maximum product concentration and the maximum-minimum gradient. Calibration datasets and prediction datasets may comprise product concentration profile data from clonal cell lines derived from the same or similar parental cell lines. For example, a calibration dataset may comprise product concentration profile data from one or more production runs of a clonal CHO cell line expressing recombinant therapeutic protein X and the prediction dataset may comprise product concentration profile data from one or more production runs of a clonal CHO cell line expressing recombinant therapeutic protein Y. The calibration dataset may also include product concentration profile data from more than one clonal cell line. The calibration dataset may include product concentration profile data from two or more different clonal cell lines from production runs using different platforms or processes.

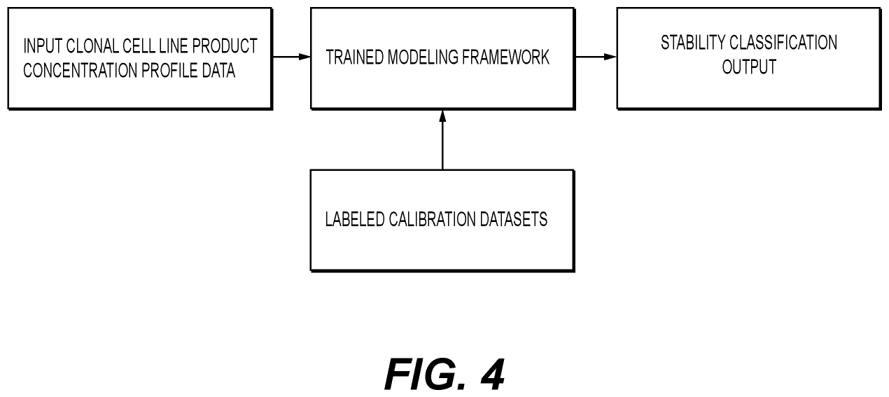

FIG. 4 provides an illustration of production stability classification of clonal cell lines using a trained modelling framework incorporating labelled calibration datasets. As may be seen, labelled calibration datasets may be provided to the modelling framework in order to train the modelling framework. At this point, the clonal cell line product concentration profile data obtained as described in connection with FIG. 1 may be input into the trained modelling framework as described in connection with FIGS. 1 through 3. A stability classification may then be output from the trained modelling framework as described in connection with FIG. 1. The skilled person will appreciate that, while not shown, this output may be used to select a clonal cell line for use in producing a therapeutic protein product.

The stability of protein production by any clonal cell line may be determined by the methods and systems described herein. The clonal cell line may be a mammalian cell line, an insect cell line, a plant cell line, a yeast cell line, a Xenopus cell line or a zebrafish cell line. The clonal cell line may be a mammalian cell line. The mammalian cell line may be a CHO (Chinese Hamster Ovary) cell line, BHK cell line, NSO cell line, Jurkat cell line, K562 cell line, HeLa cell line, HEK293 cell line, HEK293 T cell line or PerC6 cell line. The mammalian cell line may be a CHO cell line. The mammalian cell line may be a CHO cell line expressing a monoclonal antibody.

The clonal cell lines may be transfected or transformed. The clonal cell line may be transfected or transformed with a nucleic acid encoding a protein of interest. For example, the clonal cell lines may be transfected or transformed with a nucleic acid encoding a monoclonal antibody or a fragment thereof. The clonal cell lines may be transfected or transformed with a nucleic acid encoding a monoclonal antibody, a hormone, an anticoagulant, a blood factor, an interferon, a cytokine, an engineered protein scaffold, a Fc fusion protein, an enzyme, or any other suitable therapeutic protein.

The methods described herein may be computer-implemented, either in whole or in part. The method for selecting a clonal cell line may be a computer-implemented method. The modelling framework comprising multivariate latent variable modelling, multiway analysis structure, and evolving models structure may be computer-implemented.

Also provided herein is a method for predicting production stability, the method comprising:
a. inputting clonal cell line product concentration profile data into a learning model comprising a modelling framework comprising multivariate latent variable modelling, multiway analysis structure, and evolving models structure;
b. outputting, from the learning model, output indicating a production stability of a clonal cell line; and
c. identifying the clonal cell line as stable or unstable based on the output.

Also provided is a method for predicting clonal cell line production stability, the method comprising the steps of:
a. receiving clonal cell line product concentration profile data; and
b. processing the clonal cell line product concentration profile data with a system which is configured to process the clonal cell line product concentration profile data to predict clonal cell line production stability.

Also provided is a system for determining clonal cell line production stability or selecting a clonal cell line, the system comprising:
a. an input for receiving clonal cell line product concentration profile data;
b. a learning model to determine clonal cell line production stability or select a clonal cell line, the learning model comprising a modelling framework comprising multivariate latent variable modelling, multiway analysis structure, and evolving models structure;
c. one or more processors for processing clonal cell line product concentration profile data with the learning model; and
d. an output to provide an indication of clonal cell line production stability based on the processing of clonal cell line product concentration profile data by the learning model.

Also provided is a system comprising:
a. one of more processors; and
b. a non-transitory, computer readable storage medium comprising one or more programs executable by the one or more processors for performing the method described herein.

It will be understood by a person skilled in the art that certain embodiments relating to the method would be applicable to the system described herein and vice versa.

Also provided is a computer program comprising instructions which, when the program is executed by a computer or data processor(s), cause the computer to perform operations according to the method described herein.

Also provided is a computer-readable medium comprising instructions which, when executed by a computer or data processor(s), cause the computer to perform operations according to the method described herein.

The computer program or computer-readable medium may comprise instructions wherein the instructions when executed implement (a) a learning model and/or any associated function and/or (b) a system to predict clonal cell line production stability. The computer program or computer-readable medium may comprise instructions wherein the instructions when executed implement (a) a learning model and/or any associated function and/or (b) a system to determine clonal cell line production stability. The computer program or computer-readable medium may comprise instructions wherein the instructions when executed implement (a) a learning model and/or any associated function and/or (b) a system to select a stable clonal cell line. The computer program or computer-readable medium may comprise instructions wherein the instructions when executed output an output with an indication of clonal cell line production stability.

Also provided is a computer-readable data carrier having stored thereon the computer program product described herein.

### EXAMPLES

### Example 1: Scientific Validation Framework of Titre Features Fingerprint

Cell line production stability is expressed as a measure of the productivity loss over multiple runs at increasing generation numbers. Clonal cell lines tested in these production stability trials are typically defined as unstable when they display a decrease of greater than 30% in recombinant protein titre over 60 to 80 generations. Initial analysis (not shown) generated the hypothesis that modelling of titre profile variables during CLD may allow prediction of clonal cell line production stability.

Consequently, productivity titre measurements of the clonal cell lines and their evolution over time may express an early fingerprint of clonal cell line stability. To test whether clonal cell lines show this early fingerprint of stability, the full titre profiles were used as a set of multivariate inputs for the modelling framework described herein. The titre features used as regressors for the modelling approach are reported in Table 1.

TABLE 1
Titre features (i.e. production concentration profile variables)
used as regressors for the modelling approach

| # | Feature | Description |
|---|---|---|
| 1 | Mean | Mean of the titre profile |
| 2 | Standard Deviation | Standard deviation of the titre profile |
| 3 | Skewness | Shape descriptors of the titre profile distribution (degree of |
| | | asymmetry) |
| 4 | Kurtosis | Shape descriptors of the titre profile distribution (sharpness) |
| 5 | Differential | Difference between two consecutive measurements for all the titre |
| | | profile datapoints. It gives back an array of n-1 cells where n is the |
| | | number of the datapoints for a single titre profile, e.g. 7 datapoints |
| | | lead to 6 differentials (day 3-day 0, day 5-day 3 etc.). |
| 6 | Maximum | Maximum of the titre profile |
| 7 | Maximum- | Gradient of the line between the maximum and the minimum value |
| | Minimum Gradient | of the titre profile |

Data analysis approaches may be described as either “supervised” or “unsupervised”. “Supervised” analysis uses labelled input and output training datasets such that a model learns how to predict results accurately. On the other hand, “unsupervised” analysis models unlabelled datasets in order to find naturally occurring patterns in the training datasets.

A supervised learning model is effective if the set of input regressors are descriptive of the classes, i.e. the regressors have a fingerprint of the regressed variable. This can be verified by exploring the data in an unsupervised manner so that cluster-related effects can be identified in advance. The EMPLS-DA modelling approach may be translated into an unsupervised methodology by replacing the PLS-DA method with a PCA (EMPCA) where the scores diagnostic is ideal in order to visualise clusters into a lower dimensional multivariate space for all the evolving models.

EMPCA modelling was performed on two sets of data (Set A and Set B) generated from different process platforms. Traditionally, these production stability trials are performed over 4 production runs before making final stability calls on individual clonal cell lines.

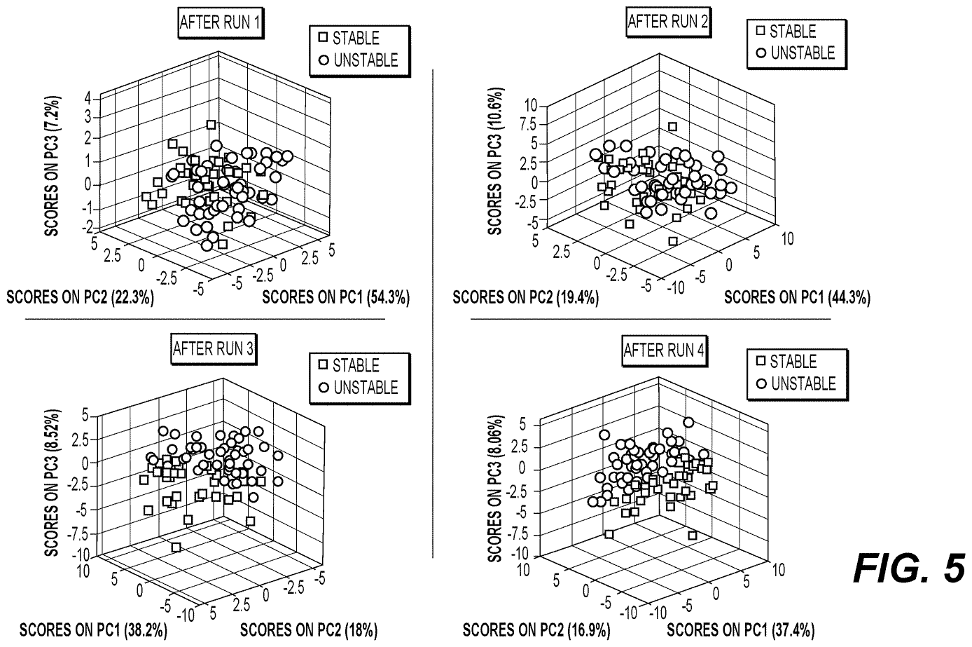

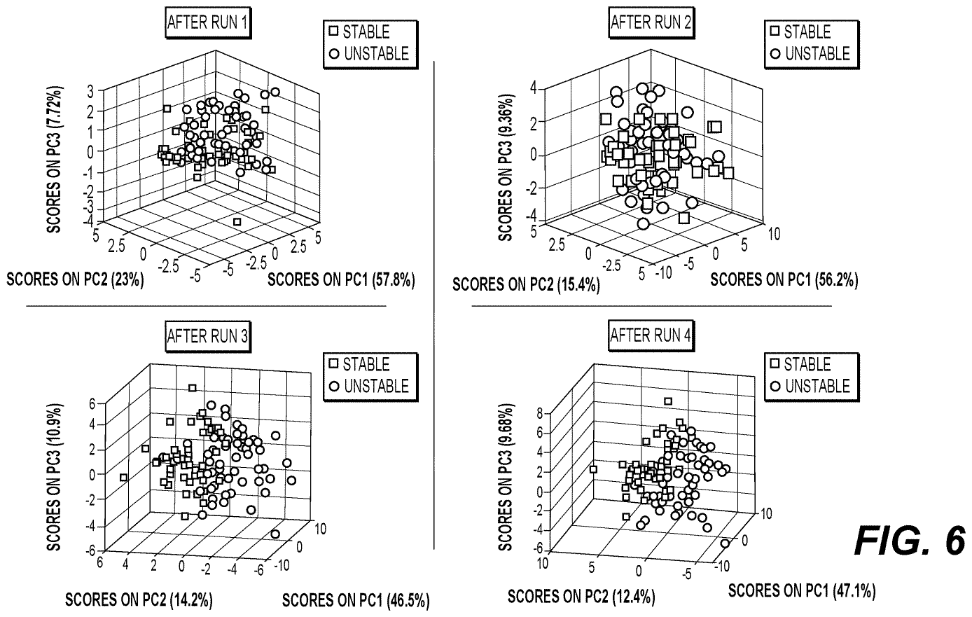

The production stability trials were run for 4 production runs over a total of 150 and 80 generations for Set A and Set B, respectively. The models built after the first two production runs do not show a strong separation of the clusters, indicating a potential weak correlation between the titre profile features used as regressors and clonal cell line stability in the first two production runs (FIGS. 5 and 6). However, the model was able to discriminate between the stable and unstable clonal cell lines in both datasets following the third production run. Accordingly, the multivariate evolving approach (EMPCA) begins to accumulate stability relevant information in a systematic way after production run 3, for that reason displaying a fingerprint of the final stability call on clonal cell lines earlier than the full set of end-point titre data that the traditional method would require. These considerations based on two different stability trial designs are indicative of the potential discrimination power of a supervised approach that would have been developed when operating in those contexts. Different designs or process platforms require a similar unsupervised analysis to understand at which production run the stability discrimination is expressed as a fingerprint of the titre feature. Platform processes that lead to different dynamic behaviours of the titre profile should be considered in separate models since the feature extraction is a description of the titre dynamic.

### Example 2: Project Testing

The performance of the developed models was tested by simulating its adoption in scenarios where final clonal cell line production stability data was available so that the performance of the models can be assessed after each production run. These simulated scenarios consider each different project to be executed consecutively so that more project data are progressively available to enlarge the calibration dataset. This approach allowed testing whether differences in the calibration dataset size and composition affect the modelling predictions.

Four scenarios were considered using different calibration datasets, prediction datasets, process platforms and stability trial end generation numbers (Table 2). The model was evaluated in these scenarios by assessing the misclassification errors rate, specificity and sensitivity after each production run in calibration as well as in prediction. The calibration results give an indication of the model robustness when calibrated, whereas the prediction results are indicative of actual predictive performance. Model calibration results with misclassification error below 30% and sensitivity greater than 70% were considered satisfactory based on a risk-based approach where sensitivity, i.e. stable predictions accuracy, is preferred over unstable predictions accuracy.

In addition to prediction of clonal cell line production stability, the developed methodology also estimates a class probability resulting in an overall confidence factor for each clonal cell line. This confidence factor is calculated using the PLS-DA methodology as described in detail herein. PLS-DA is able to describe situations where the difference between the probability of a given clonal cell line (Pᵢ) being stable (P_{i,stable}) or unstable (P_{i,unstable}) is high or low and hence predicted with high or low confidence. The model can therefore provide additional information where low confidence predictions are flagged as potentially deceiving.

TABLE 2
Simulated scenarios considered for model testing

| | Calibration | Prediction | Process | Stability trial | |
|---|---|---|---|---|---|
| Scenario | Dataset | Dataset | Platform | end criteria | |
| 1 | A | B | X | 150 | gen |
| 2 | A + B | E | X | 150 | gen |
| 3 | C | D | Y | 80 | gen |
| 4 | C + D | F | Y | 80 | gen |

The final stability calls for clonal cell lines in each project used for the assessment of the model calibration and prediction performance are shown in Table 3.

TABLE 3
Process platform, stability trial end criteria design and final stability
call for the unique clonal cell lines considered in each project

| | | Stability | | Stable | Unstable | Total |
|---|---|---|---|---|---|---|
| | Process | trial end | | clonal cell | clonal cell | clonal |
| Dataset | platform | criteria | | lines | lines | cell lines |
| A | X | 150 | gen | 29 | 17 | 46 |
| B | X | 150 | gen | 4 | 20 | 24 |
| C | Y | 80 | gen | 21 | 27 | 48 |
| D | Y | 80 | gen | 15 | 30 | 45 |
| E | X | 150 | gen | 3 | 13 | 16 |
| F | Y | 80 | gen | 19 | 5 | 24 |

In Scenario 1, the model was calibrated on 46 clonal cell lines from a single Dataset (A) using a process platform (X) measuring up to 150 cell generations in the stability trial. The calibration performance was satisfactory after the third production run with an error rate of ≤7%, ≥93% sensitivity and >94% specificity but still reasonable with 20% margin error for the first two runs (Table 4). The prediction performance had error rates of 46-54% in the first two production runs, whereas the last two runs showed good prediction performance with a misclassification error of ˜15%. The low sensitivity in prediction performance may be partially explained by the low number (4) of stable clonal cell lines in the prediction Dataset B. In-depth examination of the prediction confidence after production run 4 revealed that 3/3 of the stable misclassified clonal cell lines were flagged as low confidence, but the level of confidence for all of the stable misclassified clonal cell lines was about 60% after run 3.

TABLE 4
Model production stability classification model performance in calibration (left)
and prediction (right) for Scenario 1. Results are expressed in terms of misclassification
error, specificity and sensitivity after each production run.

| Calibration performance | | | | Prediction performance | | | |
|---|---|---|---|---|---|---|---|
| Run | Error | Specificity | Sensitivity | Run | Error | Specificity | Sensitivity |
| 1 | 20% | 71% | 86% | 1 | 54% | 50% | 25% |
| 2 | 17% | 76% | 86% | 2 | 46% | 65% | 0% |
| 3 | 7% | 94% | 93% | 3 | 17% | 100% | 0% |
| 4 | 0% | 100% | 100% | 4 | 13% | 100% | 25% |

In Scenario 2, the model was calibrated on 70 clonal cell lines from Datasets A+B. The calibration performance was consistent across production runs but remained similar in absolute values in comparison to Scenario 1 where just one dataset was used for calibration (Table 5). On the other hand, the prediction performance was considerably improved relative to Scenario 1 as very high sensitivity (=100%) and specificity (>92%) was observed after all the consecutive production runs.

TABLE 5
Model production stability classification model performance in calibration (left)
and prediction (right) for Scenario 2. Results are expressed in terms of misclassification
error, specificity and sensitivity after each production run.

| Calibration performance | | | | Prediction performance | | | |
|---|---|---|---|---|---|---|---|
| Run | Error | Specificity | Sensitivity | Run | Error | Specificity | Sensitivity |
| 1 | 24% | 78% | 73% | 1 | 19% | 92% | 33% |
| 2 | 20% | 84% | 76% | 2 | 6% | 92% | 100% |
| 3 | 13% | 92% | 82% | 3 | 6% | 92% | 100% |
| 4 | 4% | 95% | 97% | 4 | 0% | 100% | 100% |

In Scenario 3, the model was calibrated on 48 clonal cell lines from a single Dataset (C) using a process platform (Y) measuring up to 80 generations in the stability trial. The calibration performance had just a 19% misclassification error rate after the first production run, which reduced to 0% after the third and fourth runs (Table 6). The prediction performance showed the model had a sensitivity issue after the first two runs with approximately 30% misclassification error. However, the prediction performance was satisfactory after the third run with a misclassification error of 16% (7 clonal cell lines out of 45), where the 6/7 misclassified clonal cell lines were identified as low confidence predictions.

TABLE 6
Model production stability classification model performance in calibration (left)
and prediction (right) for Scenario 3. Results are expressed in terms of misclassification
error, specificity and sensitivity after each production run.

| Calibration performance | | | | Prediction performance | | | |
|---|---|---|---|---|---|---|---|
| Run | Error | Specificity | Sensitivity | Run | Error | Specificity | Sensitivity |
| 1 | 19% | 70% | 95% | 1 | 31% | 87% | 33% |
| 2 | 8% | 89% | 95% | 2 | 33% | 93% | 17% |
| 3 | 0% | 100% | 100% | 3 | 16% | 93% | 67% |
| 4 | 0% | 100% | 100% | 4 | 16% | 93% | 67% |

Finally, in Scenario 4, the model was calibrated on 93 clonal cell lines from two Datasets (C+D) using a process platform (Y) measuring up to 80 generations in the stability trial. The calibration performance slightly decreased in comparison to Scenario 3, but the prediction performance increased with run 2 behaving as an outlier which was attributable to a generation number shift (Table 7).

TABLE 7
Model production stability classification model performance in calibration (left)
and prediction (right) for Scenario 4. Results are expressed in terms of misclassification
error, specificity and sensitivity after each production run.

| Calibration performance | | | | Prediction performance | | | |
|---|---|---|---|---|---|---|---|
| Run | Error | Specificity | Sensitivity | Run | Error | Specificity | Sensitivity |
| 1 | 28% | 77% | 63% | 1 | 17% | 40% | 95% |
| 2 | 19% | 82% | 78% | 2 | 71% | 60% | 21% |
| 3 | 3% | 96% | 97% | 3 | 21% | 20% | 95% |
| 4 | 4% | 96% | 94% | 4 | 8% | 60% | 100% |

The modelling methodology accordingly uses fingerprints of clonal cell line production stability in productivity titre data to predict production stability earlier than possible using a traditional complete stability trial approach. The model described herein provides a tool for selecting the most promising stable clonal cell lines at an early stage of the cell line development process, therefore significantly reducing the time and resources required for conducting stability trials.

### Example 3: Model Testing on Live Projects

Following the initial evaluation detailed in Example 3, the decision was reached to test the modelling methodology on live projects, as well as on projects using new platforms. Tables 8, 9 and 10 show the prediction performance of the model for each project (numbered 1-5) and platform (A or B) tested. Sensitivity was always high at Run #3 (80 to 100%) and in alignment with previous results. Specificity was generally lower, sometimes appearing extremely low in the more stable platform B due to the low number of unstable clonal cell lines available (i.e. 1 unstable clonal cell line in project 3, 2 unstable clonal cell lines in project 5). The overall error rate at run #3 was low (generally ˜10% or lower). These are all metrics of satisfactory model performance in predicting clonal cell line production stability.

Project 2 behaved slightly differently from any other project analysed as it showed anomalous levels of confidence with respect to actual final production stability calculations at run #3. This resulted in very low specificity at run #3 as all unstable clonal cell lines were incorrectly predicted with low confidence, while the stable clonal cell lines were still predicted well. Therefore, a full model diagnostics was performed for project 2, which demonstrated that it is the only project where the majority of clonal cell lines showed titre profile features different and uncorrelated to the model structure for multiple Variables Important for Predictions (VIPs). VIP scores for each variable were calculated according to the equation described in Andersen & Bro 2010. Such discrepancies were found to be statistically meaningful for past projects where the model was tested and performed regularly with respect to confidence levels. The analysis was performed by comparing model residuals (both exploratory PCA and PLS) and their contributions versus VIPs for the calibrated model at Run #3. Project 2 therefore behaves as an outlier identified by diagnostics, believed to result from clonal cell lines with an atypical expression profile and very low productivity. Four out of five stable clonal cell lines were predicted correctly after Run #3 for project 2. Therefore the model still gave results valuable for stable clonal cell line selection.

The high sensitivity (98-100%) for platform B projects was likely due to the higher fraction of stable clonal cell lines generated by this particular platform and the efficient prediction of stable clonal cell lines by the model.

TABLE 8
Error rate of model for each run of projects 1-5.

| | |
|---|---|
| % Error | Run number |

| Project | Platform | 1 | 2 | 3 | 4 |
|---|---|---|---|---|---|
| 1 | A | 0.71 | 0.36 | 0.11 | 0.13 |
| 2 | A | 0.69 | 0.62 | 0.69 | 0.15 |
| 3 | B | 0.87 | 0.02 | 0.08 | 0.04 |
| 4 | B | 0.78 | 0.41 | 0.09 | 0.13 |
| 5 | B | 0.90 | 0.15 | 0.05 | 0.05 |

TABLE 9
Sensitivity rate of model for each run of projects 1-5.

| | |
|---|---|
| % Sensitivity | Run number |

| Project | Platform | 1 | 2 | 3 | 4 |
|---|---|---|---|---|---|
| 1 | A | 0.06 | 0.58 | 0.94 | 0.94 |
| 2 | A | 0.80 | 0.60 | 0.80 | 0.80 |
| 3 | B | 0.13 | 0.98 | 0.94 | 0.98 |
| 4 | B | 0.04 | 0.58 | 1.00 | 1.00 |
| 5 | B | 0.00 | 0.83 | 1.00 | 1.00 |

TABLE 10
Specificity rate of model for each run of projects 1-5.

| | |
|---|---|
| % Specificity | Run number |

| Project | Platform | 1 | 2 | 3 | 4 |
|---|---|---|---|---|---|
| 1 | A | 0.92 | 0.83 | 0.75 | 0.67 |
| 2 | A | 0.00 | 0.25 | 0.00 | 0.88 |
| 3 | B | N/A | N/A | 0.00 | 0.00 |
| 4 | B | 1.00 | 0.67 | 0.50 | 0.33 |
| 5 | B | 1.00 | 1.00 | 0.50 | 0.50 |

### Example 4: VIPs

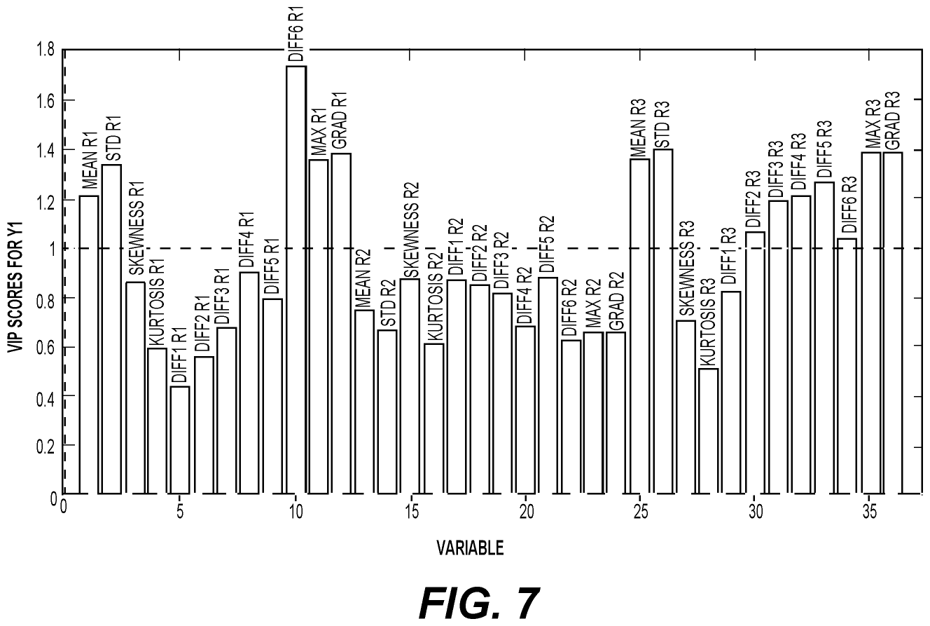

To determine the VIPs in the model for prediction of production stability, an analysis of VIPs for models built using two training datasets combined (datasets C and D in Scenario 4 of Table 2 above) was performed. A VIP score plot for each variable tested in the PLS-DA model for after run #3 is shown in FIG. 7. Variables having a VIP score greater than 1 are defined as VIPs which may have a greater influence on prediction of clonal cell line production stability. This analysis revealed 3 groups of VIPs after each production run; those with a low VIP index (close to 1), those with a medium VIP index and those with a high VIP index after each of production runs 2-4 (Table 11). Table 11 shows that the maximum of the titre profile for production run 1 (max R1), the maximum-minimum gradient for production run 1 (grad R1) and the differential6 for production run 1 (diff6 R1) were identified as variables with a high VIP score after each of production runs 2, 3 and 4. The standard deviation of the titre profile for production run 1 (std R1) was also identified as a variable with a high VIP score after production runs 3 and 4. Thus, the statistical features of the titre profile influencing prediction of clonal cell line production stability may include maximum, maximum-minimum gradient, differential and/or standard deviation.

TABLE 11
Variables Important for Prediction (VIPs)

| VIP Index | Production run 2 | Production run 3 | Production run 4 |
|---|---|---|---|
| Low | Mean R1, diff4 R2 | Diff6 R3, diff2 R3 | Skewness R1, diff4 R1, diff5 |
| | | | R1, diff3 R3, diff4 R3, diff5 |
| | | | R3, diff6 R3, mean R4, diff2 |
| | | | R4 |
| Medium | Std R1, mean R2 | Diff3 R3, diff4 R3, mean | Mean R1, mean R3, std R3, |
| | | R1, diff5 R3 | grad R3, max R3 |
| High | Diff6 R1, max R1, grad | Std R1, mean R3, max | Std R1, max R1, grad R1, |
| | R1, diff1 R2, diff2 R2, | R1, grad R1, grad R3, | diff6 R1 |
| | diff3 R2 | max R3, std R3, diff6 R1 | |

### REFERENCES

Andersen, C. M., & Bro, R. Variable selection in regression—a tutorial. Journal of Chemometrics 24 (2010): 728-737.
Brereton, Richard G., and Gavin R. Lloyd. “Partial least squares discriminant analysis: taking the magic away.” Journal of Chemometrics 28.4 (2014): 213-225.
Butler, M. & Spearman, M. The choice of mammalian cell host and possibilities for glycosylation engineering. Curr Opin Biotechnol 30, 107-112, doi: 10.1016/j.copbio.2014.06.010 (2014).
Dahodwala, H., & Lee K. H. The fickle CHO: a review of the causes, implications, and potential alleviation of the CHO cell line instability problem. Curr Opin Biotechnol 60, 128-137 https://doi.org/10.1016/j.copbio.2019.01.011 (2019).
Garcia Muñoz, S., MacGregor, J. F., Kourti, T., 2005. Product transfer between sites using Joint-Y PLS. Chemom. Intell. Lab. Syst. 79, 101-114. doi:10.1016/j.chemolab.2005.04.009
Geladi, P., & Kowalski, B. R. (1986). Partial least-squares regression: a tutorial. Analytica chimica acta, 185, 1-17.
Gower, J. C., 1975. Generalized procrustes analysis. Psychometrika 40, 33-51. doi:10.1007/BF02291478
Gunther, J. C., Baclaski, J., Seborg, D. E., Conner, J. S., 2009. Pattern matching in batch bioprocesses-Comparisons across multiple products and operating conditions. Comput. Chem. Eng. 33, 88-96. doi:10.1016/j.compchemeng.2008.07.001
Haykin, S., 2008. Neural Networks and Learning Machines, 3 edizione. ed. Prentice Hall, New York.
Jackson, J. E., 2003. A User's Guide to Principal Components. Wiley-Interscience, Hoboken, N.J.
Meneghetti, N., Facco, P., Bezzo, F., Himawan, C., Zomer, S., Barolo, M., 2016. Knowledge management in secondary pharmaceutical manufacturing by mining of data historians—A proof-of-concept study. Int. J. Pharm. 505, 394-408. https://doi.org/10.1016/j.ijpharm.2016.03.035
Nomikos, Paul, and John F. MacGregor. “Monitoring batch processes using multiway principal component analysis.” AIChE Journal 40.8 (1994): 1361-1375.
Nomikos, P., MacGregor, J. F., 1995. Multi-way partial least squares in monitoring batch processes. Chemom. Intell. Lab. Syst., InCINC '94 Selected papers from the First International Chemometrics Internet Conference 30, 97-108. https://doi.org/10.1016/0169-7439(95)000437
Ramaker, Henk-Jan, et al. “Fault detection properties of global, local and time evolving models for batch process monitoring.” Journal of Process control 15.7 (2005): 799-805.
Walczak, B., and D. L. Massart. “Dealing with missing data: Part I.” Chemometrics and Intelligent Laboratory Systems 58.1 (2001): 15-27.
Walsh, G. Biopharmaceutical benchmarks 2018. Nat Biotechnol 36, 1136-1145, doi:10.1038/nbt.4305 (2018).
Wold, S., Sjostrom, M., 1977. SIMCA: A Method for Analyzing Chemical Data in Terms of Similarity and Analogy, in: Chemometrics: Theory and Application, ACS Symposium Series. American Chemical Society, pp. 243-282. https://doi.org/10.1021/bk-1977-0052.ch012
Wold, S., Martens, H., & Wold, H. (1983). The multivariate calibration problem in chemistry solved by the PLS method. In Matrix pencils (pp. 286-293). Springer, Berlin, Heidelberg.
Wold, S. (1978). Cross-validatory estimation of the number of components in factor and principal; components models. Technometrics 20, 397-405.
Wurm, Florian M., and Maria João Wurm. “Cloning of CHO cells, productivity and genetic stability—a discussion.” Processes 5.2 (2017): 20

What is claimed is:

1. (canceled)

2. A method for producing a therapeutic protein comprising:
measuring, for a plurality of clonal cell lines, a product concentration of each clonal cell line;
determining, based on the product concentration, product concentration profile data of each clonal cell line;
inputting the product concentration profile data into a learning model comprising a modelling framework; wherein the modelling framework comprises multivariate latent variable modelling, multiway analysis structure, and evolving models structure;
generating, using the learning model, an output indicating a production stability of each clonal cell line; and
selecting, based on the output, a clonal cell, using the clonal cell line to produce a therapeutic protein.

3. The method of claim 2, wherein the multivariate latent variable modelling comprises at least one of Projection to Latent Structures (PLS) and Principal Component Analysis (PCA).

4. The method of claim 3, wherein the PLS comprises at least one of PLS-Discriminant Analysis (PLS-DA), PLS-Support-Vector Machines (PLS-SVM), PLS-Neural Networks (PLS-NN), PLS-Logistic Regression (PLS-LR), PLS-k-Nearest Neighbours (PLS-KNN), PLS-Decision Tree (PLS-DT), PLS-Naïve Bayes (PLS-NB), PLS-Random Forest (PLS-RF) and PLS-Gradient Boost (PLS-GB).

5. The method of claim 2, wherein the product concentration profile data of the clonal cell line comprises one or more of mean product concentration, standard deviation, skewness, kurtosis, differential, maximum product concentration, and maximum-minimum gradient.

6. The method of claim 2, wherein measuring, for a plurality of clonal cell lines, a product concentration of each clonal cell line comprises measuring a product concentration of each clonal cell line across a plurality of production runs.

7. The method of claim 2, wherein measuring a product concentration of each clonal cell line comprises measuring a product concentration of each clonal cell line up to a generation number of 150 generations.

8. The method of claim 6, further comprising obtaining a generation number distribution for each production run.

9. The method of claim 2, wherein the evolving models structure comprises analysing analyzing the product concentration profile data of sequential production runs.

10. The method of claim 2, wherein the clonal cell line is a mammalian cell line.

11. The method of claim 10, wherein the mammalian cell line is a CHO cell line.

12. The method of claim 2, wherein the product concentration is measured at multiple bioreactor scales.

13. (canceled)

14. The method of claim 2, wherein the product concentration comprises the concentration of a monoclonal antibody.

15. A system for determining clonal cell line production stability or selecting a clonal cell line, the system comprising:
a. an input for receiving clonal cell line product concentration profile data;
b. a learning model to determine clonal cell line production stability or select a clonal cell line, the learning model comprising a modelling framework comprising multivariate latent variable modelling, multiway analysis structure, and evolving models structure;
c. one or more processors for processing clonal cell line product concentration profile data with the learning model; and
d. an output to provide an indication of clonal cell line production stability based on the processing of clonal cell line product concentration profile data by the learning model.

16. A computer program comprising instructions which, when the program is executed by a computer or data processor(s), cause the computer to perform operations according to the method of claim 2.

17. A computer-readable medium comprising instructions which, when executed by a computer or data processor(s), cause the computer to perform operations according to the method of claim 2.

18. The program of claim 16, wherein the instructions when executed implement (a) a learning model and/or any associated function and/or (b) a system to predict clonal cell line production stability.

19. The program of claim 16, wherein the instructions when executed output an output with an indication of clonal cell line production stability.

20. A computer-readable data carrier having stored thereon the computer program of claim 16.
